(19) United States
(12) Patent Application Publication
(10) Pub. No.: US 2008/0039430 A1
(43) Pub. Date: Feb. 14, 2008

(54) Pharmaceutical Compositions For The Treatment Of Leishmaniasis

(76) Inventors: Ali Ouaissi, Boissiere (FR); Denis Sereno, Poussan (FR); Baptiste Vergnes, Montpellier (FR)

Correspondence Address: YOUNG & THOMPSON, 745 SOUTH 23RD STREET, 2ND FLOOR, ARLINGTON, VA, 22202, US

(21) Appl. No.: 11/632,845

(22) PCT Filed: Jul. 15, 2005
(86) PCT No.: PCT/EP05/07715

§ 371 Date: Apr. 19, 2007

Related U.S. Application Data
(60) Provisional application No. 60/588,802, filed on Jul. 19, 2004.

(51) Int. Cl. A61K 31/455; A61K 31/541; A61K 31/661; A61P 33/02

(52) U.S. Cl. 514/114; 514/227.8; 514/355; 514/356

(57) ABSTRACT

The present invention relates to the use of at least one compound of the following general formula (I): wherein R represents OH or NH₂, or of precursors or derivatives thereof, or of the pharmaceutically acceptable salts of the compound or of its precursors or derivatives, for the manufacture of a medicament intended for the prevention or the treatment of parasitic diseases, in particular of protozoan parasitic diseases, more particularly of leishmaniosis, and especially for the prevention or the treatment of parasitic diseases occurring in immunodepressed patients.

Protozoans belonging to the Trypanosomatidae family account for numerous pathologies afflicting man or animals.

Thus, among protozoans of the Trypanosoma genus, T. brucei and T. cruzi are for instance the etiological agents of sleep disease and Chagas disease.

Protozoans of the Leishmania genus, such as L. aethiopica, L. donovani, L. infantum, L. major, L. mexicana or L. tropica are responsible for leishmaniasis (also named leishmaniosis). Infections by these parasites are endemic in more than 88 countries. WHO estimates that more than 12 millions individuals are infected by these parasites and more than 350 millions would be exposed to infections daily. Three major forms of leishmaniosis are documented, among which the most dangerous form, visceral leishmaniosis, can have a lethal outcome in absence of treatment. This situation has worsened since the occurrence of HIV, because these infections are more frequently found as opportunistic infections in individuals afflicted by the acquired immunodeficiency syndrome (AIDS), in particular in South-West Europe. Parasites take advantage of the immunosuppressed status of the host to establish themselves or to reactivate.

Current leishmaniosis treatments are based on drugs difficult to handle, such as amphotericin B or drugs belonging to the antimonial family, which have serious side effects.

Niacin is the generic name for 2 compounds: nicotinamide (NAm) and nicotinic acid. Both were first used clinically in 1937, when these compounds were each shown to act as <<pellagra-preventive>> factor. High dose of NAm and its acid derivative nicotinic acid, are often used interchangeably to treat a number of conditions including anxiety, osteoarthritis, and psychosis. Furthermore, NAm is currently in trials as therapy to prevent cancer recurrence and insulin-dependent (Type I) diabetes (4). Beside this, activity of NAm has been evaluated in anti-mycobacterium tuberculosis studies performed during 1945-1961 and in anti-HIV studies performed from 1991 to the present (reviewed in 7).

It is an object of the present invention to provide new medicaments, lacking the drawbacks of the currently used medicaments, for the treatment of protozoan parasitic diseases, such as leishmaniosis.

Thus, the present invention relates to the use of at least one compound of the following general formula (I): 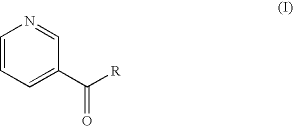
wherein R represents OH, NH₂,
or of precursors or derivatives thereof,
or of the pharmaceutically acceptable salts of said compound or of its precursors or derivatives,
for inhibiting the SIR2 protein expressed by parasites, in particular by protozoan parasites, more particularly by Leishmania, under their respective intracellular or extracellular forms.

As intended herein “precursors or derivatives” of compounds of formula (I) represent compounds which are liable to yield compounds of formula (I) in vivo or compounds which are derived from compounds of formula (I) by means of chemical modifications.

“SIR2 protein” stands for Silent Information Regulatory (SIR2) protein. SIR2 is a class III NAD-dependent deacetylase protein. It is in particular defined in Marmorstein (2004) Biochem. Transac. Society 32:904-909 or in Blander & Guarente (2004) Annu. Rev. Biochem. 73:417-435.

The expression “parasites” relates to unicellular eukaryotic organisms which are able to infect mammals and to survive and/or multiply in the infected mammal.

The present invention also relates to the use of at least one compound of the following general formula (I): 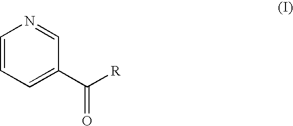
wherein R represents OH or NH₂,
or of precursors or derivatives thereof,
or of the pharmaceutically acceptable salts of said compound or of its precursors or derivatives,
for the manufacture of a medicament intended for the prevention or the treatment of parasitic diseases, in particular of protozoan parasitic diseases, more particularly of leishmaniosis, and especially for the prevention or the treatment of parasitic diseases occurring in immunodepressed patients.

As intended herein “parasitic diseases” relate to diseases caused by parasites as defined above.

Advantageously, the use of compounds of formula (I) for the prevention or the treatment of parasitic diseases is sound, since numerous bioavailability studies have assessed that high plasma concentrations of these compounds, e.g. 2.3 mM, could be achieved without serious side effects.

In a preferred embodiment of the above defined use of a compound of formula (I), R represents OH, said compound corresponding to niacin (vitamin B3), of the following formula (II):

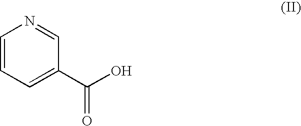

In another preferred embodiment of the above defined use of a compound of formula (I), R represents NH₂, said compound corresponding to nicotinamide, of the following formula (III):

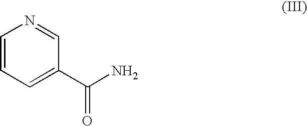

According to another preferred embodiment of the above defined use, the medicament is suitable for an administration of the compound of formula (I) by oral, intravenous, topical or intralesional route.

As intended herein “intralesional” means that the medicament is suitable to be administered at the sites of parasite-caused skin lesions of patients, in particular in case of Leishmania infections.

According to a particularly preferred embodiment of the above defined use, the medicament is suitable for an administration of the compound of formula (I) at a unit dose of about 10 mg to about 10 g, in particular of about 1 g to about 6 g.

According to another particularly preferred embodiment of the above defined use, the medicament is suitable for an administration of the compound of formula (I) at a dosage of about 5 mg/m²/day to about 5 g/m²/day, in particular of about 500 mg/m²/day to about 3 g/m²/day.

In another preferred embodiment of the above defined use, the compound of formula (I) in association with at least one anti-parasitic compound, such as a compound selected from:

miltefosin, antimonials, amphotericin B, benznidazol, nifurtimox, paromomycin, pentamidin and its derivatives, arsenic derivatives, melarsopol and difluoromethylornithin.

Advantageously, the association of a compound of formula (I) with an anti-parasitic compound has additive or synergic effects which enables a diminished administration of said anti-parasitic compound and thus diminished side effects.

The present invention also relates to a pharmaceutical composition comprising as active substances:

at least one compound of the following general formula (I): 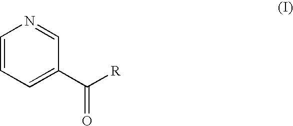
wherein R represents OH or NH₂,
or precursors or derivatives thereof, or of the pharmaceutically acceptable salts of said compound or of its precursors or derivatives, and

at least one anti-parasitic compound, such as a compound selected from:

miltefosin, antimonials, amphotericin B, benznidazol, nifurtimox, paromomycin, pentamidin and its derivatives, arsenic derivatives, melarsopol and difluoromethylornithin,

in association with a pharmaceutically acceptable carrier.

In a particular embodiment of the above defined pharmaceutical composition, R represents OH, the compound of formula (I) hence corresponding to niacin (vitamin B3).

In another particular embodiment of the above defined pharmaceutical composition, R represents NH₂, the compound of formula (I) hence corresponding to nicotinamide.

According to a preferred embodiment, the above defined pharmaceutical composition is suitable for an administration by oral intravenous, topical or intralesional route.

According to another preferred embodiment, the above defined pharmaceutical composition is suitable for the administration of the compound of formula (I) at a unit dose of about 10 mg to about 10 g, in particular of about 1 g to about 6 g.

According to yet another preferred embodiment, the above defined pharmaceutical composition is suitable for the administration of the compound of formula (I) at a dosage of about 5 mg/m²/day to about 5 g/m²/day, in particular of about 500 mg/m²/day to about 3 g/m²/day.

The present invention also relates to products containing:

at least one compound of the following general formula (I): 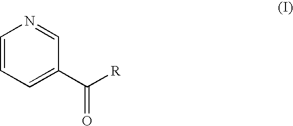
wherein R represents OH or NH₂,
or precursors or derivatives thereof,
or the pharmaceutically acceptable salts of said compound or of its precursors or derivatives, in association with

at least one anti-parasitic compound, such as a compound selected from:

miltefosin, antimonials, amphotericin B, benznidazol, nifurtimox, paromomycin, pentamidin and its derivatives, arsenic derivatives, melarsopol and difluoromethylornithin, as a combined preparation for simultaneous, separate or sequential use in the prevention or the treatment of parasitic diseases, in particular of protozoan parasitic diseases, more particularly of leishmaniosis, and especially for the prevention or the treatment of parasitic diseases occurring in immuno-depressed patients.

In a preferred embodiment of the above defined products, R represents OH, the compound of formula (I) hence corresponding to niacin (vitamin B3).

In another preferred embodiment of the above defined product, R represents NH₂, the compound of formula (I) hence corresponding to nicotinamide.

The present invention also relates to a method for the prevention or the treatment of parasitic diseases, in particular of protozoan parasitic diseases, more particularly of leishmaniosis, and especially for the prevention or the treatment of parasitic diseases occurring in immuno-depressed patients, characterized in that at therapeutically effective amount of at least one compound of the following general formula (I): 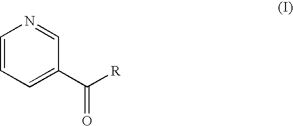
wherein R represents OH, NH₂,
or of precursors or derivatives thereof,
or of the pharmaceutically acceptable salts of said compound or of its precursors or derivatives,
is administered to a patient in need thereof.

In a preferred embodiment of the above defined method, R represents OH, the compound of formula (I) hence corresponding to niacin (vitamin B3).

In another preferred embodiment of the above defined method, R represents NH₂, the compound of formula (I) hence corresponding to nicotinamide.

According to a particular embodiment of the above defined method, the compound of formula (I) is administered by oral intravenous, topical or intralesional route.

According to another particular embodiment of the above defined method, the compound of formula (I) is administrated at a unit dose of about 10 mg to about 10 g, in particular of about 1 g to about 6 g,

According to yet another particular embodiment of the above defined method, the compound of formula (I) is administered at a dosage of about 5 mg/m²/day to about 5 g/m²/day, in particular of about 500 mg/m²/day to about 3 g/m²/day.

In another preferred embodiment of the above defined method, the compound of formula (I) is administered in association with at least one anti-parasitic compound, such as a compound selected from:

miltefosin, antimonials, amphotericin B, benznidazol, nifurtimox, paromomycin, pentamidin and its derivatives, arsenic derivatives, melarsopol and difluoromethylornithin.

### DESCRIPTION OF THE FIGURES

FIG. 1A, FIG. 1B, FIG. 1C, and FIG. 1D

FIG. 1A represents the mean number of viable leishmania at the promastigote stage (vertical axis, ×10⁶/ml) as a function of time (horizontal axis, days), in presence of no nicotinamide (NAm) (control, white circles), 10 mM NAm (grey circles), or 20 mM NAm (black circles).

FIG. 1B represents the mean number of viable axenically grown amastigotes leishmania (vertical axis, ×10⁶/ml) as a function of time (horizontal axis, days), in presence of no nicotinamide (NAm) (control, white squares), 10 mM NAm (grey squares), or 20 mM NAm (black squares).

FIG. 1C represents the mean percentage of YOPRO-1-positive axenically grown amastigotes (i.e. apoptotic cells) (vertical axis) as a function of time (horizontal axis, days) in presence of 25 mM Nam (squares), 50 mM Nam (diamonds), or 100 mM Nam (circles). Results are expressed as a mean of a triplicate experiment.

FIG. 1D represents the parasitic index (vertical axis) as a function of NAm concentration (horizontal axis, mM). Results are representative of one over two experiments carried out in sextuplate (one star (*) corresponds to P<0.05, two stars (**) correspond to P<0.005, and three stars (***) correspond to P<0.001).

FIG. 2A and FIG. 2B

FIG. 2A represents the NAD-dependent deacetylase activity of the SIRT1 enzyme expressed as the fluorescence at 355 nm to fluorescence at 460 nm ratio (F355/F460) (vertical axis, counts) for (from left to right) a control assay without NAD (first histogram), a control assay with NAD (second histogram), an assay with 5 mM NAm (third histogram), an assay with 20 mM NAm (fourth histogram), an assay with 5 mM NAc (fifth histogram), an assay with 20 mM NAc (sixth histogram) and a control assay (seventh histogram).

FIG. 2B represents the NAD-dependent deacetylase activity detected in leishmania expressed as the relative fluorescence at 355 nm to fluorescence at 460 nm ratio (F355/F460) (vertical axis, counts) for leishmania carrying an empty pTEX plasmid (first histogram), leishmania carrying a plasmid expressing LmSIR2 (pTEX-LmSIR2) (second histogram), leishmania carrying a plasmid expressing LmSIR2 (pTEX-LmSIR2) in presence of 5 mM NAm and leishmania carrying a plasmid expressing LmSIR2 (pTEX-LmSIR2) in presence of 50 μM pentamidine. Results are given as a mean of two duplicate experiments.

FIG. 3A and FIG. 3B

FIG. 3A represents the percentage of growth inhibition (vertical axis) as a function of NAm concentration (horizontal axis, mM) for wild type (WT) leishmania (black histogram), leishmania carrying a pTEX-LmSIR2 plasmid (vertically hatched histogram) or leishmania carrying a control pTEX plasmid (horizontally hatched histogram).

FIG. 3B represents the percentage of YOPRO-1 positive cells (vertical axis) as a function of NAm concentration (horizontal axis, mM) for wild type (WT) leishmania (black histogram), or leishmania carrying a pTEX-LmSIR2 plasmid (vertically hatched histogram). Results are expressed as mean value of a quadruplate experiments.

### EXAMPLES

### Example 1

The growth of Leishmania amastigotes and promastigotes was followed in axenic culture conditions in the presence or absence of NAm.

A cloned line of L. infantum (MHOM/MA/67/ITMAP-263) was used in all experiments. Each subculture was initiated at 5×10⁵ parasites/ml of medium. Axenically grown amastigote forms of L. infantum were maintained at 37° C. with 5% CO₂ by weekly subpassages in a cell-free medium called MAA/20 (medium for axenically grown amastigotes) in 25-ml flasks, as previously described (10). Promastigote forms were maintained at 26° C. by weekly subpassage in SDM 79 medium supplemented with 10% foetal calf serum (FCS) and 100 units/ml penicillin and 100 μg/ml streptomycin. Nicotinamide (SIGMA, St Louis) was added at the appropriate concentration and the mean number of viable parasites determined using FACs analysis, as previously described (11).

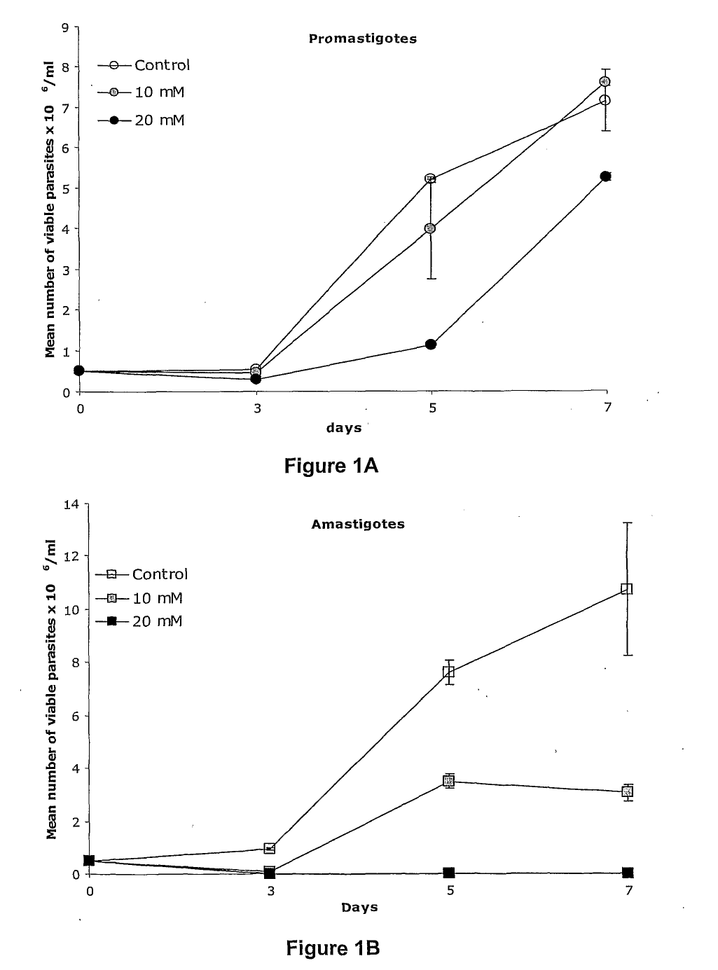

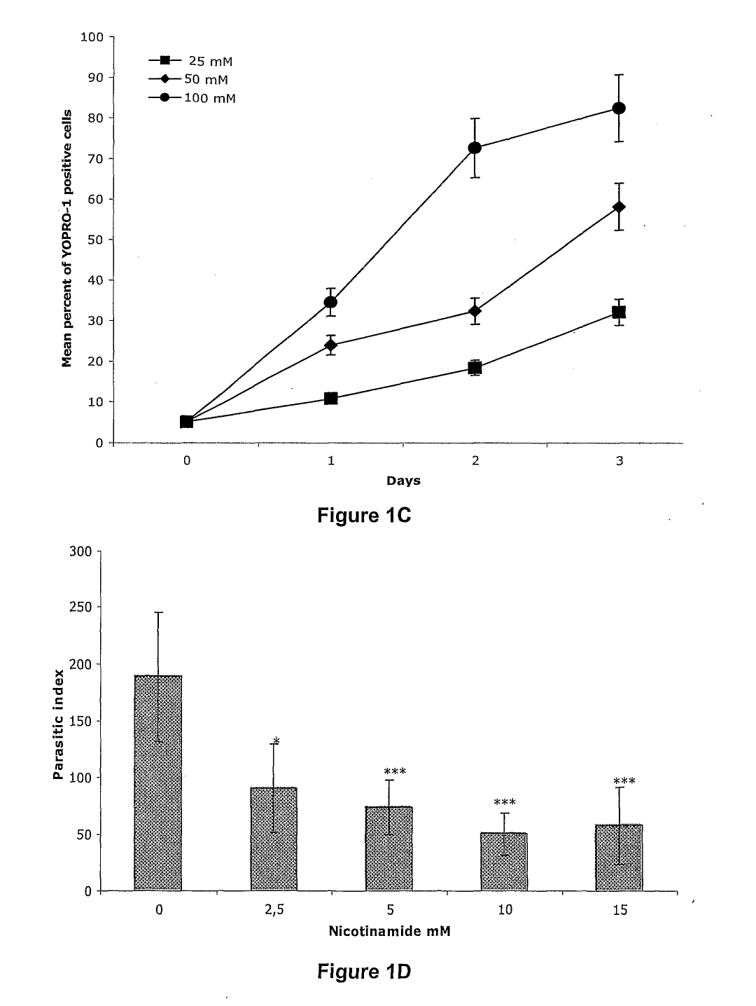

As shown in FIGS. 1A and 1B, NAm strongly inhibited the proliferation of both promastigotes and amastigotes with promastigote forms showing less sensitivity to NAm than amastigotes. At 20 mM NAm, the capacity of axenic amastigotes to proliferate was virtually completely abrogated, whereas a delay in the growth of promastigotes occurred. The growth inhibitory activity of nicotinamide was not restricted to L. infantum since L. amazonsensis amastigotes were also found to be sensitive to the activity of NAm. Furthermore, it was found that the acid derivative of NAm, the nicotinic acid (NicotAc or NAc), exerted a growth inhibitory activity towards Leishmania parasites, although at higher concentrations.

### Example 2

The nature of NAm-induced amastigotes growth arrest was then investigated

Cells were seeded at 5×10⁵ parasites/ml and NAm was added at various concentrations ranging from 25 to 100 mM. After 24, 48 and 72 hours of incubation aliquots (10⁶ parasites) were collected, washed and incubated for 10 min with 10 μM of YOPRO-1, an apoptotic cell marker (Molecular probes). The mean percentage of YOPRO-1 positive cells was determined as previously described (9). At concentrations higher than 25 mM, NAm exerted a strong dose-dependent leishmanicidal activity against axenic amastigote, as demonstrated by the occurrence of YOPRO-1 positive cells. Maximal effect was observed after 3 days of culture in the presence of 100 mM of NAm (FIG. 1C).

Having observed that NAm induced axenic amastigotes death, it was of interest to examine its effect on intracellular amastigotes proliferation.

In a first series of experiments, THP-1 monocytes were incubated during 3 days with various concentrations of NAm and the growth and viability of cells were recorded. Up to 10 mM of NAm, no effect on cell growth and viability was observed. In contrast, 20 mM NAm inhibited the proliferation of THP-1 monocyte by about 45% in agreement with the values recorded for other cell types: SupT1 and PBLs cells (6).

Thus, THP-1 differentiated macrophages were infected with stationary phase amastigotes at a host cell-parasite ratio of 5:1. After 4 hours, non adherent parasites were removed and nicotinamide was added to the medium at the appropriate concentration. After 3 days of incubation time, cells were fixed with methanol and stained with giemsa. Parasitic index PI (mean percentage of infected macrophages X number of amastigotes per macrophage) was determined. As shown in FIG. 1D, NAm significantly inhibited the in vitro proliferation of intracellular amastigote. Maximal activity was observed with 10 mM of NAm. At this concentration a reduction of almost 70% of PI was observed. Interestingly, at low dosage 2.5 mM NAm is also able to significantly inhibit intracellular amastigote proliferation when compared to control non treated cultures (p<0.05).

### Example 3

It has been recently demonstrated that NAm is a substrate of sir2-like enzymes in vitro (5). Therefore, complementary experiments were conducted in order to examine whether NAm could interfere with Leishmania deacetylase activity in vitro. To test this possibility, a commercially available “cyclex SIR2 assay kit” and SIRT1 as a standard enzyme (MBL, Japan) were used.

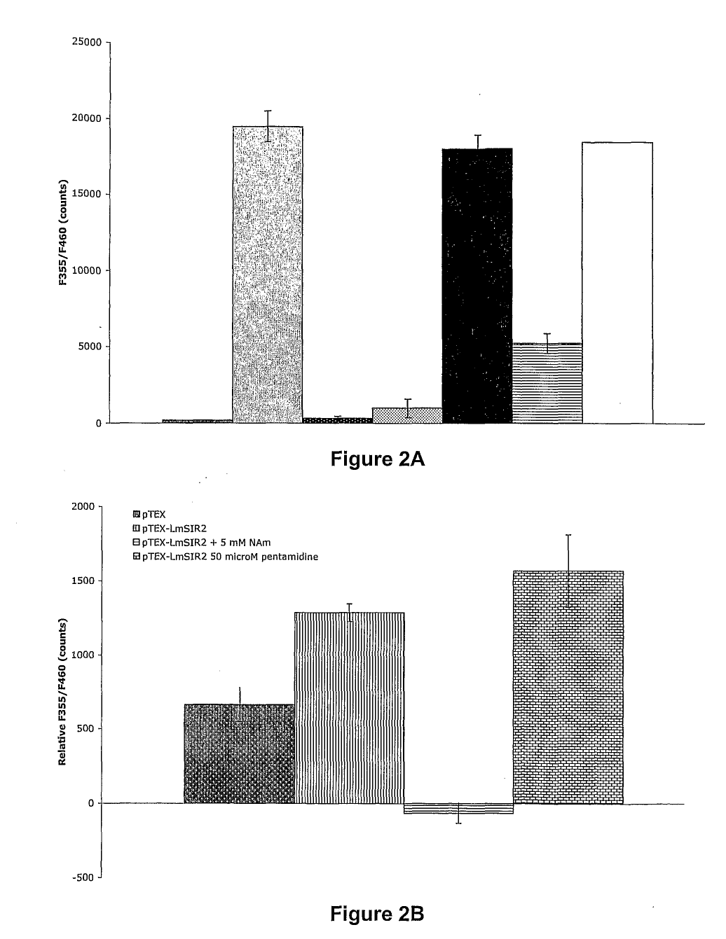

As shown in FIG. 2A, the deacetylase activity of SIRT1 is strictly dependent on the presence of NAD, addition of 5 mM or 20 mM NAm in the assay almost completely abrogated the enzymatic activity of SIRT1. In contrast, 5 mM of NicotAc had no significant effect, in agreement with the data reported by other investigators (2), whereas 20 mM of NicotAc showed a significant effect.

Having established a standard inhibitory assay, the effect of NAm was then examined on the NAD-dependent deacetylase activity contained in Leishmania extracts from mutant parasites carrying extra copies of LmSIR2 gene (pTEX-LmSIR2) or empty plasmid DNA (pTEX) (11).

Briefly, 2 10⁵ parasites were collected and washed two times with PBS 0.01M pH 7.2 and incubated in a lysis solution (100 mM Tris-HCl, 150 mM NaCl, 1 mM EDTA, 1% NP40, 5 μM Trichostatin A, pH 8.8), cells were then centrifuged for 20 min at 10 000 rpm at 4° C. Deacetylase activity in the presence or absence of 200 μM NAD was measured. Results are expressed as relative F355/F460 counts=F355/F460 counts in the presence of NAD—F355/F460 counts in the absence of NAD. This allowed to discriminate between fluorescence due to the action of LmSIR2 to fluorescence due to the presence of compounds which could interfere with the test. As shown in FIG. 2B, parasites overexpressing LmSIR2 had more NAD-dependent deacetylase activity than parasites carrying the empty pTEX vector. 5 mM of NAm significantly inhibited the NAD dependent deacetylase activity detected in parasites overexpressing LmSIR2 (FIG. 2B).

### Example 4

In yeast and C. elegans, SIR2 is a limiting component of longevity (reviewed in 3) and NAm is able to accelerate yeast ageing by inhibiting SIR2 in vivo (2). In the protozoan parasite L. infantum, amastigotes carrying extracopies of LmSIR2 (LiSIR2) gene, when maintained under normal axenic culture conditions, showed striking increase in the survival due to an inherent resistance to apoptosis-like death, leading to a longer stationary phase of growth (11).

To further examine the possible correlation between the level of SIR2 expression and the sensitivity/resistance to NAm-induced Leishmania amastigotes death, NAm was added to cultures of mutant L. infantum amastigotes which overexpress LmSIR2 or carrying the empty pTEX plasmid as controls.

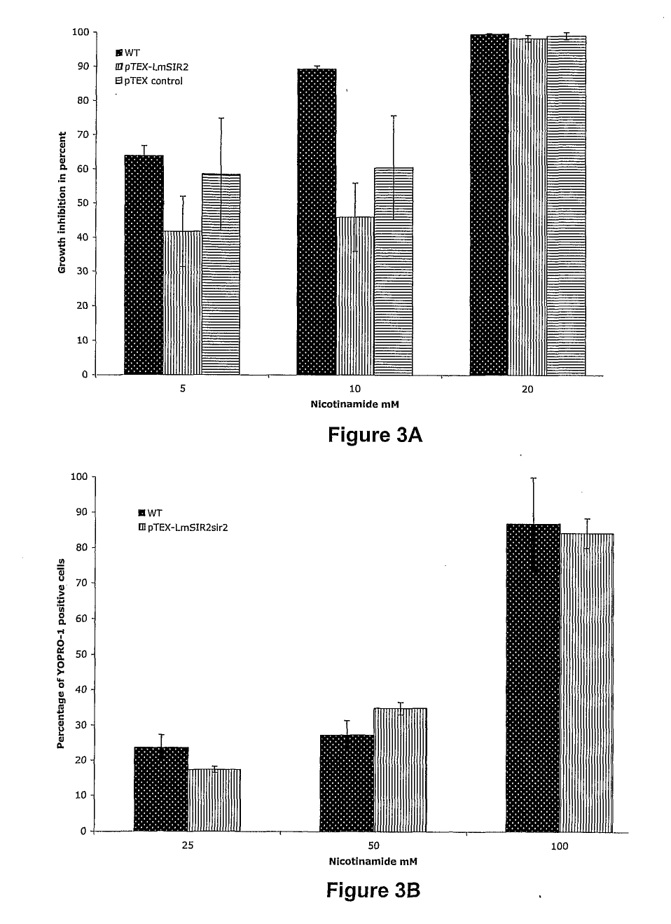

As shown in FIGS. 3 A and 3B, adding extra copies of LmSIR2 to amastigotes did not confer significant resistance to NAm-induced death. Thus, even if the NAD-dependent deacetylase activity of LmSIR2 is readily inhibited by NAm and that LmSIR2 play a role in the survival of Leishmania amastigotes it should represent only one of the target of NAm mediated cell growth arrest.

The microbicidal mechanism of action of NAm is not currently known. Its activity may come to be understood as that of an indirect antimicrobial that has primarily a prohost effect. Among the reasons to suggest effect is the body of literature that reports an immunodulatory role for nicotinamide in a wide variety of experimental systems (8, 7). Moreover, antioxydant and cryoprotective effect of NAm is well documented (12).

Thus, the present invention represents the first report showing the anti-parasitic activity of NAm. Furthermore, although NAm could inhibit the NAD-dependent deacetylase activity of SIR2-like enzymes, its main target in Leishmania seems not to be LmSIR2. In fact Leishmania possesses two other SIR2 related members whose function and localization are currently unknown. Implication of this protein family in the survival of Leishmania parasite has to be investigated. It can be hypothesized that one or all of them are essential for the parasite survival, and that their inhibition leads to the parasite death. Alternatively, other essential physiological functions would be the targets for NAm. The concentration of NAm and Nicotinic acid found to inhibit the intracellular growth of Leishmania infantum (IC50 inferior to 2.5 mM) are far higher than those found in whole blood (about 45 μM) but is closer to the plasmatic concentration of nicotinamide achievable (0.7 to 2.3 mM) in patient treated with accelerated radiotherapy for head and Neck cancer (1).

In conclusion, nicotinamide is an inexpensive and orally available agent without significant side effects. Since nicotinamide and its derivatives are potentially beneficial components, leishmaniasis will benefit from therapeutic use of such components, optionally in combination with anti-parasitic drugs.

### REFERENCES

1. Bernier J., M. R. L. Strztford, J Deneckamp, M. F. Dennis, S. Bieri, F. Hagen, O Kocagöncü, M. Bolla and A. Rojas. 1998. Pharmacokinetics of nicotinamide in cancer patients treated with accelerated radiotherapy: the experience of the Co-operative group of radiotherapy of the european organization for research and treatment of cancer. Radiation & Oncology. 48: 123-133.
2. Bitterman K. J., R. M. Anderson, R. Y. Cohen, M. Latorre-Esteves and D. A. Sinclair. 2002. Inhibition of silencing and accelerated aging by nicotinamide, a putative negative regulator of yeast sir2 and human SIRT1. J Biol. Chem. 277: 45099-107.
3. Imai S., F. B. Jonhson, R. A. Marciniak, M. McVey, P. U. Park and L. Garante. 2000. Sir2: an NAD-dependent histone deacetylase that connects chromatin silencing, metabolism, and aging. Cold. Spring. Harb. Symp. Quant. Biol. 65:297-302.
4. Kaanders J H., L. A. Pop, H. A. Marres, I. Bruaset, F. J. van den Hoogen, M. A. Merkx and A. J. van der Kogel. 2002. ARCON: experience in 215 patients with advanced head-and-neck cancer. Int J Radiat Oncol Biol Phys. 52: 769-78.
5. Landry J., A. Sutton, S. T. Tafrov, R. C. Heller, J. Stebbins, L. Pillus and R. Sternglanz. 2000. The silencing protein SIR2 and its homologs are NAD-dependent protein deacetylases. Proc Natl Acad USA. 97: 5807-5811.
6. Murray M. F and A Srinivasan. 1995. Nicotinamide inhibits HIV-1 in both acute and chronic in vitro infection. Biochem Biophys Res Commun. 210: 954-9.
7. Murray M. F. 2003. Nicotinamide: an oral antimicrobial agent with activity against both Mycobacterium tuberculosis and human immunodeficiency virus. Clin Infect Dis. 36: 453-60.
8. Otsuka A, T., J. Hanafusa, J. Miyagawa, N. Kono and S. Tarui. 1991. Nicotinamide and 3-aminobenzamide reduce interferon-gamma-induced class II MHC (HLA-DR and DP) molecule expression on cultured human endothelial cells and fibroblast. Immunopharmacol immunotoxicol. 13: 263-280. Proc Natl Acad Sci USA. 97: 5807-11.
9. Sereno D., P. Holzmuller, I. Mangot, G. Cuny, A. Ouaissi and J. L Lemesre. 2001. Antimonial-mediated DNA fragmentation in Leishmania infantum amastigotes. Antimicrob Agents Chemother. 45:2064-9.
10. Sereno, D and J. L. Lemesre. 1997. Axenically cultured amastigote forms as an in vitro model for investigation of antileishmanial agents. Antimicrob Agents Chemother. 41: 972-6.
11. Vergnes B., D. Sereno, N. Madjidian-Sereno, J. L. Lemesre and A. Ouaissi. 2002. Cytoplasmic SIR2 homologue overexpression promotes survival of Leishmania parasites by preventing programmed cell death. Gene. 21: 139-50.
12. Yang, J and J. D Adams. 2004. Nicotinamide and its pharmacological properties for clinical therapy. Drug Design Review. 1 : 43-52.

1-16. (canceled)

17. A method for the prevention or treatment of parasitic diseases;
comprising administering to a subject in need thereof an effective amount of at least one compound of the following general formula (I): 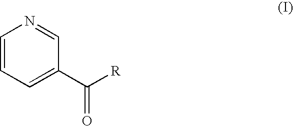
wherein R represents OH or NH2, or of precursors or derivatives thereof, or of the pharmaceutically acceptable salts of said compound or of its precursors or derivatives.

18. The method according to claim 17, of a compound of general formula (I), wherein R represents OH, said compound corresponding to niacin (vitamin B3), of the following formula (II):

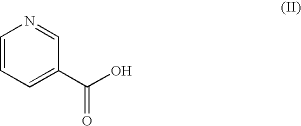

19. The use according to claim 17, of a compound of general formula (I), wherein R represents NH2, said compound corresponding to nicotinamide, of the following formula (III):

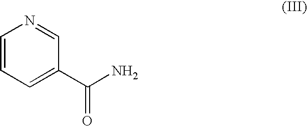

20. The method according to claim 17, wherein the medicament is suitable for an administration by oral, intravenous, topical or intralesional route.

21. The method according to claim 17, wherein the medicament is suitable for an administration of the compound of formula (I) at a unit dose of about 10 mg to about 10 g, in particular of about 1 g to about 6 g.

22. The method according to claim 17, wherein the medicament is suitable for an administration of the compound of formula (I) at a dosage of about 5 mg/m2/day to about 5 g/m2/day, in particular of about 500 mg/m2/day to about 3 g/m2/day.

23. The method according to claim 17, wherein the compound of formula (I) is in association with at least one anti-parasitic compound, such as a compound selected from: miltefosin, antimonials, amphotericin B, benznidazol, nifurtimox, paromomycin, pentamidin and its derivatives, arsenic derivatives, melarsopol and difluoromethylornithin.

24. A pharmaceutical composition comprising as active substances:—at least one compound of the following general formula (I): 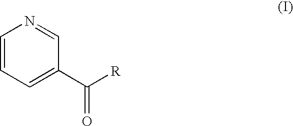
wherein R represents OH or NH2, or precursors or derivatives thereof, or of the pharmaceutically acceptable salts of said compound or of its precursors or derivatives, and—at least one anti-parasitic compound, such as a compound selected from: miltefosin, antimonials, amphotericin B, benznidazol, nifurtimox, paromomycin, pentamidin and its derivatives, arsenic derivatives, melarsopol and difluoromethylornithin,—in association with a pharmaceutically acceptable carrier.

25. The pharmaceutical composition according to claim 24, wherein R represents OH, the compound of formula (I) hence corresponding to niacin (vitamin B3).

26. The pharmaceutical composition according to claim 24, wherein R represents NH2, the compound of formula (I) hence corresponding to nicotinamide.

27. The pharmaceutical composition according to claim 24 in a form for administration by oral, intravenous, topical or intralesional route.

28. The pharmaceutical composition according to claim 24, wherein the compound of formula (I) is at a unit dose of about 10 mg to about 10 g.

29. The pharmaceutical composition according to claim 24, wherein the compound of formula (I) is at a dosage of about 5 mg/m2/day to about 5 g/m2/day.

30. A product comprising at least one compound of the following general formula (I): 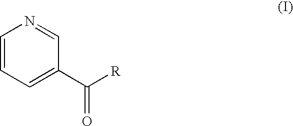
wherein R represents OH, NH2, or precursors or derivatives thereof, or of the pharmaceutically acceptable salts of said compound or of its precursors or derivatives, in association with—at least one anti-parasitic compound, such as a compound selected from: miltefosin, antimonials, amphotericin B, benznidazol, nifurtimox, paromomycin, pentamidin and its derivatives, arsenic derivatives, melarsopol and difluoromethylornithin, as a combined preparation for simultaneous, separate or sequential use in the prevention or the treatment of parasitic diseases, in particular of protozoan parasitic diseases, more particularly of leishmaniosis, and especially for the prevention or the treatment of parasitic diseases occurring in immunodepressed patients.

31. The product according to claim 30, wherein R represents OH5 the compound of formula (I) hence corresponding to niacin (vitamin B3).

32. The product according to claim 30, wherein R represents NH2, the compound of formula (I) hence corresponding to nicotinamide.
